(19) United States
(12) Patent Application Publication
(10) Pub. No.: US 2024/0295538 A1
(43) Pub. Date: Sep. 5, 2024

(54) RAW MILK MEASURING APPARATUS AND METHOD OF MANUFACTURING THE SAME

(71) Applicant: ADVANTEST CORPORATION, Tokyo (JP)

(72) Inventors: Makoto YAMAZAKI, Saitama (JP); Hoshito TAGUCHI, Gunma (JP); Tomio KUROSAWA, Saitama (JP)

(73) Assignee: ADVANTEST CORPORATION, Tokyo (JP)

(21) Appl. No.: 18/574,967

(22) PCT Filed: Mar. 9, 2022
(86) PCT No.: PCT/JP2022/010241

§ 371 Date: Dec. 28, 2023

(30) Foreign Application Priority Data
Aug. 11, 2021 (JP) 2021-131287

(51) Int. Cl. G01N 33/04; G01N 11/02

(52) U.S. Cl. CPC G01N 33/04; G01N 11/02

(57) ABSTRACT

A raw milk measuring apparatus includes a flow path. a surfactant. and an electrode. Raw milk flows through the flow path. The surfactant is placed on a side wall of the flow path. The electrode is at least partially disposed within the flow path. The surfactant is at least partially placed upstream the electrode.

### TECHNICAL FIELD

The present invention relates to measuring raw milk.

### BACKGROUND ART

There has conventionally been known measuring milk. For example, there has been known a technique in which milk is poured into a first container with surfactant applied on the inner surface thereof and reaches a second container provided with an electrode, where the number of somatic cells is measured (see FIG. 1 of Patent Literature 2, for example). However, in accordance with the related art described in Patent Literature 2, the second container must be cleaned after the measurement.

On the other hand, there has been known a sensor for which milk is poured into a flow path with surfactant applied on the bottom surface thereof and reaches an electrode provided in the flow path, where the number of somatic cells is measured (see FIG. 2 of Patent Literature 1, for example). In accordance with the related art described in Patent Literature 1, the sensor is a single-use one, which saves time and effort to clean it after the measurement.

### CITATION LIST

### Patent Literature

Patent Literature 1: Japanese Patent Application Publication No. 2010-230363

Patent Literature 2: Japanese Patent Application Publication No. 2010-256231

### SUMMARY OF THE INVENTION

### Technical Problem

However, in accordance with the related art described in Patent Literature 1, the bottom surface of the flow path must be applied with surfactant. This means that precise adjustment of the position to apply the surfactant is required with respect to the bottom surface of the flow path.

It is hence an object of the present invention to save effort for adjustment of the position to place surfactant upon measuring raw milk.

### Means for Solving the Problem

According to the present invention, a raw milk measuring apparatus, includes: a flow path through which raw milk flows; a surfactant placed on a side wall of the flow path; and an electrode at least partially disposed within the flow path, wherein the surfactant is at least partially placed upstream the electrode.

According to the present invention, raw milk flows through a flow path. A surfactant is placed on a side wall of the flow path. An electrode is at least partially disposed within the flow path. The surfactant is at least partially placed upstream the electrode.

According to the present invention, the raw milk measuring apparatus may further include: a top portion having an inlet and an outlet for the raw milk; a bottom portion on which the electrode is disposed; and an intermediate layer disposed between the top portion and the bottom portion in which the side wall is formed.

According to the raw milk measuring apparatus of the present invention, the intermediate layer may be adhered to the top portion and the bottom portion.

According to the present invention, a method of manufacturing the raw milk measuring apparatus includes: placing the surfactant on the side wall; and disposing the intermediate layer between the top portion and the bottom portion.

### BRIEF DESCRIPTION OF DRAWINGS

FIG. 1 is an exploded perspective view of a raw milk measuring apparatus 1 according to an embodiment of the present invention;

FIG. 2 is a cross-sectional view of the raw milk measuring apparatus 1 according to the embodiment of the present invention;

FIG. 3 is a plan view of an intermediate layer 120 according to the embodiment of the present invention;

FIGS. 4 (a) and 4 (b) is a cross-sectional view of the intermediate layer 120 according to the embodiment of the present invention taken along IV-IV before (FIG. 4 (a)) and after (FIG. 4 (b)) application of surfactant 122; and

FIG. 5 is a plan view of the intermediate layer 120 according to the embodiment of the present invention when disposed on a bottom portion 130.

### Modes for Carrying out the Invention

A description will now be given of an embodiment of the present invention referring to drawings.

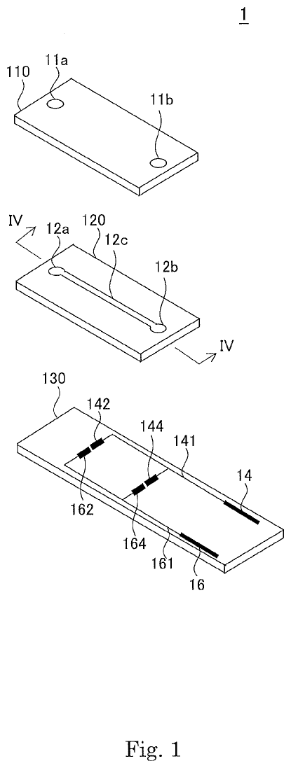

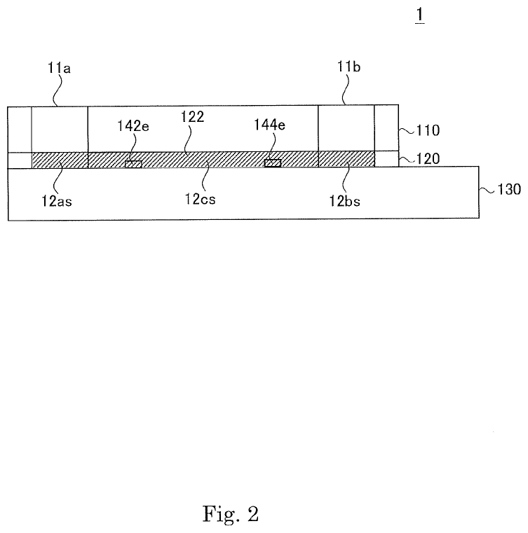

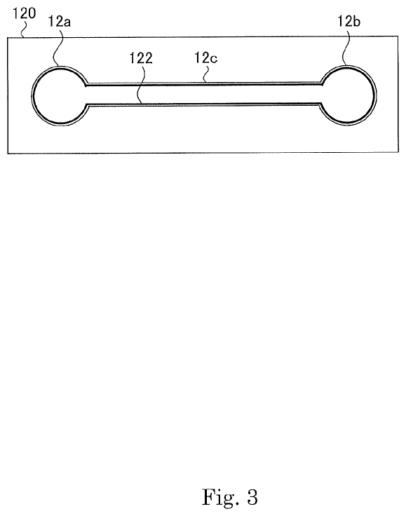

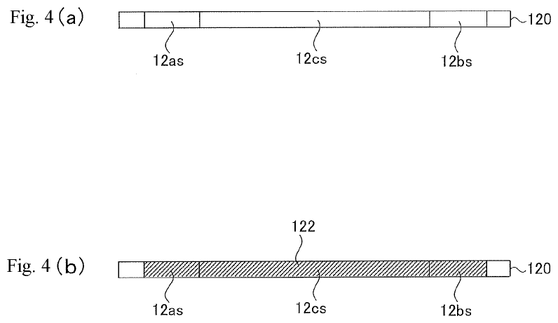

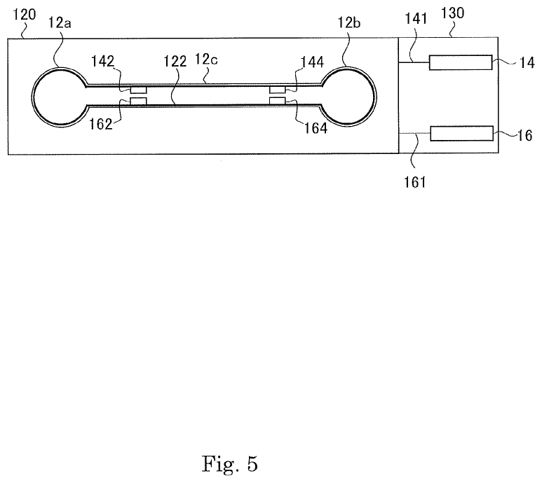

FIG. 1 is an exploded perspective view of a raw milk measuring apparatus 1 according to an embodiment of the present invention. FIG. 2 is a cross-sectional view of the raw milk measuring apparatus 1 according to the embodiment of the present invention. FIG. 3 is a plan view of an intermediate layer 120 according to the embodiment of the present invention. FIGS. 4 (a) and 4 (b) is a cross-sectional view of the intermediate layer 120 according to the embodiment of the present invention taken along IV-IV before (FIG. 4 (a)) and after (FIG. 4 (b)) application of surfactant 122. FIG. 5 is a plan view of the intermediate layer 120 according to the embodiment of the present invention when disposed on a bottom portion 130.

The raw milk measuring apparatus 1 includes a top portion 110, an intermediate layer 120, and a bottom portion 130. The top portion 110 has an inlet 11a and an outlet 11b for raw milk. Raw milk poured through the inlet 11a flows through flow paths 12a, 12b, 12c and exits through the outlet 11b. It is noted that the inlet 11a and the outlet 11b are holes penetrating through the top portion 110. Electrodes 142, 144, 162, 164 are disposed on the bottom portion 130. The intermediate layer 120 is disposed between and adhered to the top portion 110 and the bottom portion 130. It is noted that the bottom portion 130 is lager than the intermediate layer 120 to have a portion not covered with the intermediate layer 120.

Raw milk flows through the flow paths 12a, 12b, 12c that are formed in the intermediate layer 120. The flow paths 12a, 12b, 12c are holes penetrating through the intermediate layer 120. The flow path 12a is disposed beneath the inlet 11a, while the flow path 12b is disposed beneath the outlet 11b. The flow path 12c connects the flow path 12a and the flow path 12b.

Surfactant 122 is placed on (e.g. applied to) side walls 12as, 12bs, and 12cs of the respective flow paths 12a, 12b, and 12c that are formed in the intermediate layer 120.

The electrodes 142, 144, 162, 164 are at least partially disposed within the flow path 12c (see FIG. 5). The surfactant 122 is at least partially placed upstream the electrodes 142, 144, 162, 164 (i.e. nearer the inlet 11a and the flow path 12a).

It is noted that the raw milk measuring apparatus 1 may be manufactured by placing the surfactant 122 on the side walls 12as, 12bs, 12cs and further disposing the intermediate layer 120 between the top portion 110 and the bottom portion 130.

A terminal 14, a wire 141, a terminal 16, and a wire 161 is disposed on the bottom portion 130. The terminals 14, 16 are disposed in a portion of the bottom portion 130 not covered with the intermediate layer 120 (see FIG. 5).

The terminal 14 is connected through the wire 141 to the electrodes 142, 144. The terminal 16 is connected through the wire 161 to the electrodes 162, 164. A voltage is applied between the terminals 14 and 16. For example, the terminal 14 has a positive potential, while the terminal 16 is grounded. This causes a voltage to be applied between the electrode 142 and the electrode 162 and also between the electrode 144 and the electrode 164.

It is noted that an end face 142e of the electrode 142 (see FIG. 2), an end face 144e of the electrode 144 (see FIG. 2), an end face of the electrode 162, and an end face of the electrode 164 are disposed within the flow path 12c.

Next will be described an operation of the raw milk measuring apparatus 1 according to the embodiment of the present invention.

First of all, surfactant 122 is applied to the side walls 12as, 12bs, 12cs. Further, an intermediate layer 120 is disposed between and adhered by adhesive to the top portion 110 and the bottom portion 130. A raw milk measuring apparatus 1 is thus manufactured.

Viscous substances such as DNA and cellular matrix are present within white blood cells in the raw milk. Raw milk is poured into and through the inlet 11a of the raw milk measuring apparatus 1 to reach the flow path 12a. The raw milk that has reached the flow path 12a then flows through the flow path 12c toward the flow path 12b.

Here, in the flow path 12a and the flow path 12c, the surfactant is at least partially placed upstream the electrodes 142, 162. The surfactant then disrupts the cell membrane of white blood cells in the raw milk, which causes viscous substances such as DNA and cellular matrix to be ejected into the raw milk. This causes the raw milk to have high viscosity.

The raw milk flowing through the flow path 12c passes between the electrodes 142 and 162 and further between the electrodes 144 and 164 to reach the flow path 12b. The raw milk further passes through the flow path 12b to exit through the outlet 11b.

Here, the higher the viscosity of the raw milk, at the lower velocity the raw milk flows. At time point t1 when the raw milk passes between the electrodes 142 and 162, the resistance between the electrode 142 and the electrode 162 becomes lower. At time point t2 when the raw milk passes between the electrodes 144 and 164, the resistance between the electrode 144 and the electrode 164 becomes lower. Accordingly, based on t2−t1=Δt and the distance between the electrodes 142 and 144 (or between the electrodes 162 and 164), the flow velocity and viscosity of the raw milk and therefore the number of white blood cells in the raw milk can be measured.

In accordance with the embodiment of the present invention, the raw milk measuring apparatus 1 can be a single-use one. Additionally, upon measuring raw milk with the raw milk measuring apparatus 1, the position adjustment of the surfactant 122 can be completed only by placing (applying) the surfactant 122 on the side walls 12as, 12bs, 12cs and adhering the intermediate layer 120 to the bottom portion 130 (there is no need to apply the surfactant 122 precisely in the vicinity of the electrodes 142, 144, 162, 164 on the bottom portion 130), whereby it is possible to save effort for the adjustment.

### DESCRIPTION OF REFERENCE NUMERALS

1 Raw Milk Measuring Apparatus
110 Top Portion
11a Inlet
11b Outlet
120 Intermediate Layer
12a, 12b, 12c Flow Path
12as, 12bs, 12cs Side Walls
122 Surfactant
130 Bottom Portion
142, 144, 162, 164 Electrodes
142e, 144e End Face
14, 16 Terminal
141, 161 Wire

1. A raw milk measuring apparatus, comprising:
a flow path through which raw milk flows;
a surfactant placed on a side wall of the flow path; and
an electrode at least partially disposed within the flow path, wherein
the surfactant is at least partially placed upstream the electrode.

2. The raw milk measuring apparatus according to claim 1, further comprising:
a top portion having an inlet and an outlet for the raw milk;
a bottom portion on which the electrode is disposed; and
an intermediate layer disposed between the top portion and the bottom portion in which the side wall is formed.

3. The raw milk measuring apparatus according to claim 2, wherein
the intermediate layer is adhered to the top portion and the bottom portion.

4. A method of manufacturing the raw milk measuring apparatus according to claim 2, comprising:
placing the surfactant on the side wall; and
disposing the intermediate layer between the top portion and the bottom portion.

5. A method of manufacturing the raw milk measuring apparatus according to claim 3, comprising:
placing the surfactant on the side wall; and
disposing the intermediate layer between the top portion and the bottom portion.
